(19) United States
(12) Patent Application Publication
(10) Pub. No.: US 2010/0228099 A1
(43) Pub. Date: Sep. 9, 2010

(54) DEVICE FOR PREDICTING PROGNOSIS OF PATIENTS WHO UNDERGO PEG OPERATION, METHOD FOR PREDICTING PROGNOSIS OF PATIENTS WHO UNDERGO PEG OPERATION AND COMPUTER READABLE MEDIUM

(75) Inventors: Toshifumi Hibi, Tokyo (JP); Tetsuro Takayama, Tokyo (JP)

Correspondence Address: SUGHRUE-265550, 2100 PENNSYLVANIA AVE. NW, WASHINGTON, DC, 20037-3213, US

(73) Assignees: Toshifumi HIBI, Tokyo (JP); Tetsuro TAKAYAMA, Tokyo (JP); OTSUKA PHARMACEUTICAL FACTORY , INC., Naruto-shi ,Tokushima (JP)

(21) Appl. No.: 12/677,932

(22) PCT Filed: Mar. 19, 2009
(86) PCT No.: PCT/JP2009/055476

§ 371 Date: Mar. 12, 2010

(30) Foreign Application Priority Data
May 7, 2008 (JP) 2008-121326 2008

(51) Int. Cl. A61B 5/00

(52) U.S. Cl. 600/300

(57) ABSTRACT

The present invention provides a device for predicting prognosis of a patient who undergoes PEG, which allows a doctor to obtain predictive results sufficient to judge whether or not to perform the PEG (percutaneous endoscopic gastrostomy) for the patient. This device includes: a prognosis prediction expression storage section 14 configured to store a prognosis prediction expression, the prognosis prediction expression being calculated by applying prediction input factors and prediction output factors about a first patient who already underwent the PEG, to ANN (artificial neural network); and a processing section 16 configured to: receive diagnosis input factors about a second patient, for judging whether or not to perform the PEG on the second patient; calculate diagnosis output factors in response to the diagnosis input factors based on the prognosis prediction expression; and output the calculated diagnosis output factors.

### TECHNICAL FIELD

This invention relates to a predicting device and a predicting method, which predict prognosis of patients who undergo percutaneous endoscopic gastrostomy, and a computer readable medium causing a computer to perform the predicting method.

### BACKGROUND OF THE INVENTION

PEG (Percutaneous Endoscopic Gastrostomy) is a technique which is given to a patient who, for example, has a disorder in the deglutition function and therefore has a difficulty in orally ingesting nutrition, and the object of this technique is to prevent deglutition pneumonia and improve nutritive condition of said patient, by carrying out administration of nourishment into the stomach directly through the stomach and abdominal wall.

Since PEG is technically easy and management after operation is also convenient, it is a technique frequently used for patients. On the other hand, depending on the disease which caused a disorder in the deglutition function, such as cerebrovascular accidents, malignant diseases, deglutition pneumonia, dementia or degenerative diseases, there are a risk of causing death by PEG and a risk of inducing a complication with the above-mentioned disease even when PEG is performed to a patient, so that there is a case in which long-term survival of said patient cannot be obtained. Accordingly, an opinion has been proposed that PEG should be performed to a patient whose survival for three months or more after the execution can be expected.

Based on the above-mentioned opinion, examinations have been carried out on the influences of various prediction factors upon the prognosis of patient who underwent PEG. For example, it has been reported in Non-patent Reference 1 that when PEG was performed to a patient having a high serum albumin value, prognosis of said patient was good. Also, it has been reported in Non-patent References 2 and 3 that when PEG was performed to patients having progressive dementia, dementia and the like diseases as complications, said patients showed poor prognosis in comparison with patients having no complications. In the present state, doctors are judging whether the PEG is performed or not, based on such reports.

Non-patent Reference 1: Friedenberg F, Jensen G, Gujral N, Braitman L E and Levine G M, Serum albumin is predictive of 30-day survival after percutaneous endoscopic gastrostomy, Jpen., 1997, March-April, 21 (2), 72-4.

Non-patent Reference 2: Sanders D S, Carter M J, D′Silva, James G, Bolton RP and Bardhan K D, Survival analysis in percutaneous endoscopic gastrostomy feeding: a worse outcome in patients with dementia, The American Journal of Gastroenterology, 2000, June, 95(6), 1472-5.

Non-patent Reference 3: Rimon E, Kagansky N and Levy S, Percutaneous endoscopic gastrostomy; evidence of different prognosis in various patient subgroups, Age Aging, 2005, July, 34 (4), 353-7

### DISCLOSURE OF THE INVENTION

### Problems that the Invention is to Solve

However, it must be said that the above-mentioned reports alone are not sufficient for doctors to judge whether or not the PEG is performed. This is because the items reported in the above-mentioned Non-patent References are on the influence exerted by a certain one predictive factor (serum albumin value in the case of Non-patent Reference 1, and the presence or absence of certain diseases in the case of Non-patent References 1 and 2). When prognosis of a patient who underwent PEG is predicted, it is needles to say that it is necessary to carry out versatile analyses based on various predictive factors which represent conditions of said patient.

Studies have so far been attempted on the prediction method which predicts prognosis of a patient who underwent PEG, based on two or more predictive factors. However, the previous studies are analyzing techniques in which two or more predictive factors are applied to linear discriminant analysis, and in these days that nonlinear properties of almost all phenomena in the living body were verified, it cannot be said that even the predictive results based on the above-mentioned previous studies are sufficient for doctors to judge whether or not PEG should be performed.

The invention has been made by taking the above-mentioned situations into consideration, and its object is to provide a device for predicting prognosis of a patient who undergoes PEG (percutaneous endoscopic gastrostomy), which can produce predictive results sufficient enough for doctors to judge whether or not the PEG should be performed to the patient, a method for predicting prognosis of a patient who undergoes PEG, and a computer readable medium causing a computer to perform the predicting method.

### Means for Solving the Problems

In order to achieve the object of the invention, a device that predicts prognosis of a patient who undergoes PEG (percutaneous endoscopic gastrostomy) according to the invention, is characterized by the following (1) to (4).

(1) The device comprises:

a prognosis prediction expression storage section that stores a prognosis prediction expression, said prognosis prediction expression being calculated by applying prediction input factors and prediction output factors about a first patient who already underwent the PEG, to ANN (artificial neural network); and

a processing section that inputs diagnosis input factors about a second patient, for judging whether or not to perform the PEG on the second patient, calculates diagnosis output factors in response to the diagnosis input factors by referring to the prognosis prediction expression stored in the prognosis prediction expressing storage section and then outputs the calculated diagnosis output factors.

(2) The device according to claim (1), further comprises:

a prediction factor data base that stores the prediction input factors and the prediction output factors about the first patient; and

an analytical program storage section that stores a program for calculating the prognosis prediction expression by applying the prediction input factors and the prediction output factors about the first patient to the ANN, and

wherein

the processing section calculates the prognosis prediction expression calculated by applying the prediction input factors and the prediction output factors about the first patient stored in the prediction factor data base, by referring to the program stored in the analytical program storage section, and then stores the calculated prognosis prediction expression in the prognosis prediction expression storage section.

(3) The device according to claim (1) or (2), at least one item of the prediction output factors and the diagnosis output factors is the number of survival days after the PEG, or the presence or absence of onset of deglutition pneumonia after the PEG.

(4) The device according to claim (3), the prediction input factors and the diagnosis input factors includes, as items, at least age, sex, the presence or absence of a cerebrovascular accident, the presence or absence of a malignant disease, the presence or absence of deglutition pneumonia before gastrostomy, the presence or absence of dementia, the presence or absence of a degenerative disease, an amount of serum total protein, an amount of serum albumin and an amount of hemoglobin.

In order to achieve the object of the invention, a method for predicting prognosis of a patient who undergoes PEG (percutaneous endoscopic gastrostomy) according to the invention is characterized by the following (5) to (6).

(5) The method comprises the steps of:

inputting diagnosis input factors about a first patient, for judging whether or not to perform the PEG on the first patient;

calculating diagnosis output factors in response to the diagnosis input factors input in said input step, by referring to a prognosis prediction expression calculated by applying prediction input factors and prediction output factors about a second patient who already underwent PEG to ANN (artificial neural network); and

outputting the diagnosis output factors calculated in the calculation step.

(6) The method according to claim (5), further comprises:

calculating the prognosis prediction expression in which the prediction input factors and the prediction output factors about the second patient are applied to ANN.

In addition, in order to achieve the object of the invention, a computer readable medium according to the invention is characterized by the following (7).

(7) A computer readable medium stores a program causing a computer to execute the method according to claim (5) or (6).

According to the device according to the above-mentioned (1) or (2), it is possible to inform a doctor of a prediction result which is sufficient for judging whether or not to perform PEG for the patient.

According to the device according to the above-mentioned (3), it is possible to inform a doctor of factors important for the doctor in judging whether or not to perform PEG.

According to the device according to the above-mentioned (4), it is possible to highly accurately calculate factors which are important for the doctor in judging whether or not to perform PEG.

According to the method according to the above-mentioned (5) or (6), it is possible to inform a doctor of a prediction result which is sufficient for judging whether or not to perform PEG for the patient.

According to the computer readable medium according to the above-mentioned (7), it is possible to inform a doctor of a prediction result which is sufficient for judging whether or not to perform PEG for the patient.

### ADVANTAGE OF THE INVENTION

According to the device for predicting prognosis of a patient who undergoes PEG, the method for predicting prognosis of a patient who undergoes PEG and the computer readable medium causing a computer to execute the prediction method in the present invention, by predicting prognosis of a patient who underwent PEG based on two or more prediction factors, it is possible to predict the number of survival days of the patient after PEG and a possibility of a complication with the disease which caused a disorder in deglutition with a high accuracy. As a result of this, it is possible to inform doctor of a prediction result which is sufficient enough for judging whether or not to perform PEG for the patient.

### BRIEF DESCRIPTION OF THE DRAWINGS

FIG. 1 is a hardware block diagram of a prediction device according to an embodiment of the invention.

FIG. 2 is a flow chart showing processings by the prediction device according to the embodiment of the invention.

### DESCRIPTION OF THE REFERENCE NUMERALS

11 Input section
12 Prediction factor data base
13 Analytical program storage section
14 Prognosis prediction formula storage section
15 Display section
16 Processing section

### BEST MODE FOR CARRYING OUT THE INVENTION

The following illustratively describes a device for predicting prognosis of post-PEG patients (to be referred simply to as prediction device hereinafter) of an embodiment of the invention with reference to drawings.

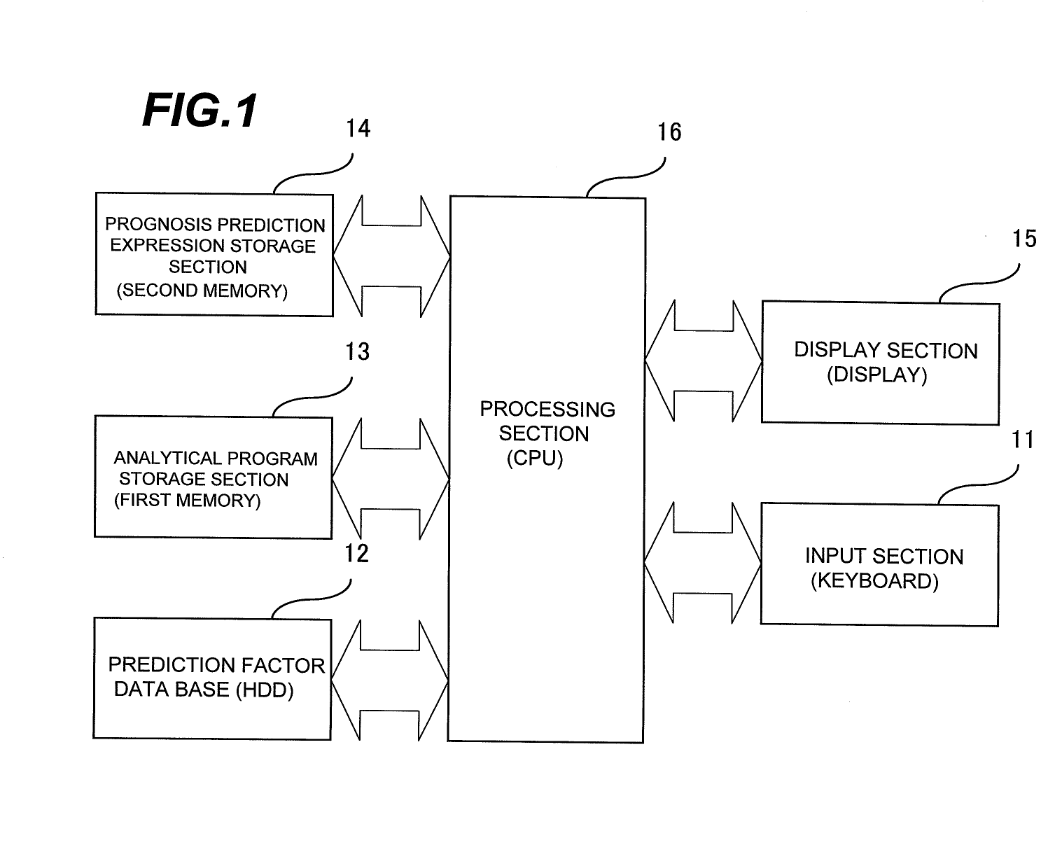

A hardware block diagram of a prediction device of the embodiment of the invention is shown in FIG. 1. The prediction device of an embodiment of the invention includes an Input section 11, a prediction factor data base 12, an analytical program storage section 13, a prognosis prediction formula storage section 14, a display section 15 and a processing section 16. When the prediction device of the embodiment of the invention is configured by a general purpose PC for example, the Input section 11 is realized by a key board, a mouse, a ten-key keypad and the like various input interface, the prediction factor data base 12 is realized by a hard disc drive (HDD), the analytical program storage section 13 and the prognosis prediction formula storage section 14 are realized by RAM (random access memory), the display section 15 is realized by a CRT display, a liquid crystal display and the like various input devices, and the processing section 16 is realized by CPU (central processing unit). The device which realizes the Input section 11, prediction factor data base 12, analytical program storage section 13, prognosis prediction formula storage section 14, display section 15 and processing section 16 is not limited to the above-mentioned one, and a device which can perform functions of the respective sections, as described below, can be appropriately used.

Firstly, regarding an analytical program which is stored in the analytical program storage section 13, an outline of its algorithm is described below. Artificial neural networks (ANN) are applied to the algorithm which predicts prognosis of a post-PEG patient. ANN is a learning system which is based on a calculation technique which simulates a neurological processing by the human brain, and is useful in modeling a system in which both of dependent variable and independent variable are present. ANN patternizes relationships which are present between input values and output values, with further high accuracy, by patternizing and learning the relationships between the input values and the output values, and further by recognizing a new relationship which is present between input values and output values and then patternizing and learning the new relationship. ANN is roughly divided into a phase which patternizes, based on already-known input values and output values, a relationship between the input values and the output values (corresponds to the “patternizing phase” which is described later) and a phase which outputs an output value corresponding to a new input value, by referring to the patternized relationship (corresponds to the “diagnosis phase” which is described later), when the new input vale is input.

The above-mentioned “input value” and “output value” regarding the prediction device of the embodiment of the invention represents, for example, the following parameters shown in Table 1. In this connection, a parameter corresponding to an “input value” is called “input factor”, and a parameter corresponding to an “output value” is called “output factor”. Also, the above-mentioned “already-known input value and output value” are generally referred to as “prediction factors”, and further among the “prediction factors”, the “already-known input value” is called “prediction input factor” and the “already-known output value” is called “prediction output factor”. In addition, the above-mentioned “new input value and an output value corresponding to the new input value” are generally referred to as “diagnosis factors”, and further among the “diagnosis factors”, the “new input value” is called “diagnosis input value” and the “output value corresponding to the input value” is called “diagnosis output value”.

TABLE 1

| Input factors | Output factors |
|---|---|
| Age (years) | |
| Sex (male or female) | |
| The presence or absence of | |
| cerebrovascular accidents | |
| The presence or absence of | |
| cerebral bleeding | |
| The presence or absence of | |
| cerebral infarction | |
| The presence or absence of | |
| cerebral contusion | |
| The presence or absence of | |
| deglutition pneumonia | |
| before PEG | |
| The presence or absence of | |
| malignant tumor | |
| The presence or absence of | |
| gastric cancer | |
| The presence or absence of | The number of survival days |
| esophageal carcinoma | (days) |
| The presence or absence of | The presence or absence of |
| large bowel cancer | post-PEG deglutition pneumonia |
| The presence or absence of | The presence or absence of |
| mouth cancer | diarrhea |
| The presence or absence of | The presence or absence of |
| dementia | bleeding |
| The presence or absence of | The presence or absence of |
| cerebrovascular dementia | self removal |
| The presence or absence of | Death of old age |
| senile dementia of | |
| Alzheimer type | |
| The presence or absence of | The presence or absence of |
| a degenerative disease | wound infection |
| The presence or absence of | |
| Parkinson disease | |
| The presence or absence of | |
| OPCA | |
| The presence or absence of | |
| depression | |
| The presence or absence of | |
| loss appetite | |
| WBC (g/dl) | |
| Hb (g/dl) | |
| TP (g/dl) | |
| Alb (g/dl) | |
| TC (g/dl) | |
| Kind of nasal nourishment | |

According to the prediction device of the embodiment of the invention, the prediction factors shown in Table 1 are stored in the prediction factor data base 12. That is, for each patient who already underwent PEG, prediction input factors before PEG for said patient and prediction output factors after PEG for said patient are made into a data base and stored. When prediction factors are stored in the prediction factor data base 12, the processing section 16 stores the values input by the device user through the input section 11 such that they correspond to each patient.

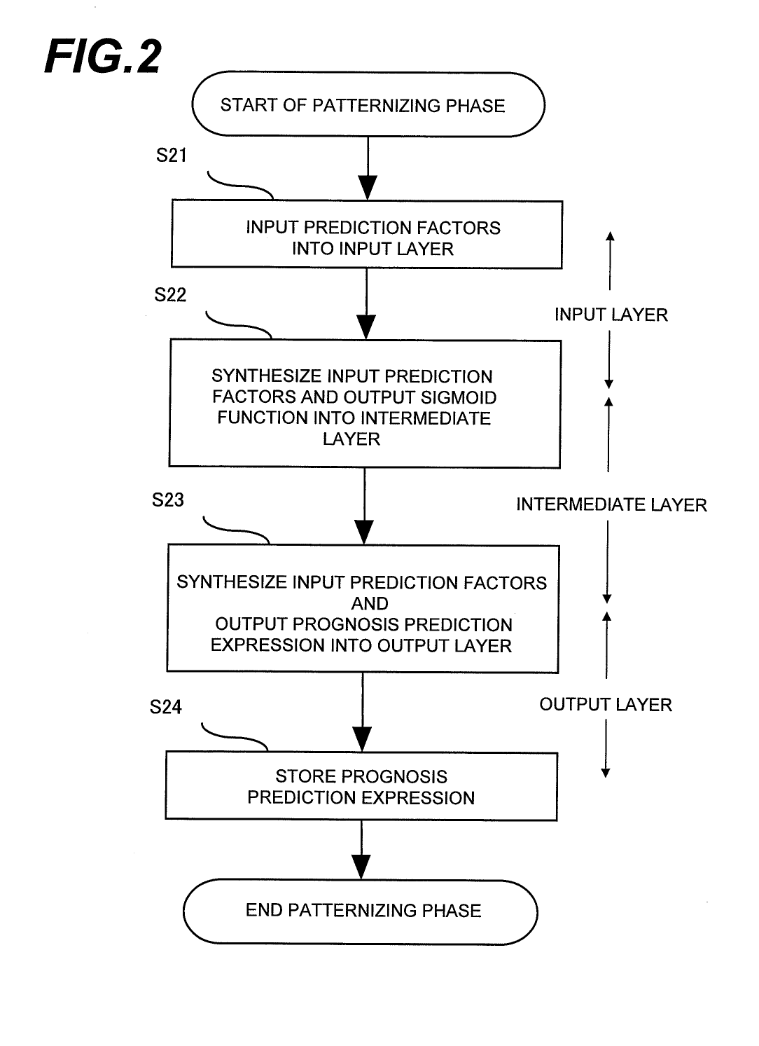

The processing section 16 firstly patternizes the prediction factors of two or more patients stored in the prediction factor data base 12, based on the artificial neural network algorithm-applied program extracted in the analytical program storage section 13. In the following, an example of the illustrative patternization by the prediction device of the embodiment of the invention is described with referring to a flow chart of FIG. 2, which shows processing by the prediction device of the embodiment of the invention.

A hierarchical type artificial neural network (ANN) is made up of an input layer, an intermediate layer and an output layer, a unit corresponding to a nerve cell is present in each layer, and information is propagated from the input layer to the output layer via the intermediate layer. When prediction factors are input from the prediction factor data base 12 into the input layer (step 21), each unit in the input layer and intermediate layer synthesizes the prediction factors from the input layer by the following Expression (1) and outputs the sigmoid function of Expression (2) into the intermediate layer as an operation function (step 22).

$$\begin{matrix}{y}_{j} = \sum {w}_{i , j} {x}_{i} & ( 1 ) \\ f ( {y}_{j} ) = \frac{1}{1 + exp ( - α {y}_{j} )} & ( 2 )\end{matrix}$$

In this case, W_{i,j} is a weight between the next layer unit j and the front layer i unit, and Xi is an output from the front layer. The f(yⱼ) is transferred as the output value to the next layer. The α is slope of the sigmoid function. ANN is able to approximate a non-linear quantitative relationship between factors and characteristics via a process called “learning” which means optimization of the W_{i,j} value.

For example, information is propagated using the matrix W_{i,j} shown in Table 2. However, the matrix of W_{i,j} shown below is an example obtained by said learning.

TABLE 2
outcome Y1
N(hidden) 6

| 1st-layer | X1 | x2 | x3 | x4 | x5 |
|---|---|---|---|---|---|
| hu 1 | 0.101606 | −0.38785 | 0.194975 | 3.154559 | −0.63533 |
| hu 2 | 0.18058 | 0.818023 | −4.06913 | −7.52947 | 0.817051 |
| hu 3 | 0.219609 | −0.01235 | −0.57573 | 3.212002 | −1.90118 |
| hu 4 | −0.4719 | −0.49822 | −4.17598 | 1.445426 | 4.426148 |
| hu 5 | −0.17136 | 0.772233 | −5.03082 | −0.96078 | 1.095073 |
| hu 6 | −0.31844 | 0.95863 | 8.155914 | 1.013073 | 0.653753 |
| | x6 | x7 | x8 | x9 | x10 |
| hu 1 | −4.97056 | 1.431707 | 2.281485 | 2.310622 | 1.358225 |
| hu 2 | 0.471236 | −8.50971 | −0.95146 | −3.02889 | 0.143723 |
| hu 3 | −7.39203 | 14.72972 | 1.180469 | 4.925159 | 0.33013 |
| hu 4 | 10.03522 | 0.727034 | −2.07688 | −1.59155 | 6.025189 |
| hu 5 | 5.947987 | 5.473299 | −4.99163 | −1.73274 | −3.04714 |
| hu 6 | 6.884651 | 0.888955 | 4.936559 | 1.145654 | 4.335977 |

However, the outcome y1 shows the number of survived days after PEG, and the number of hidden layers as the intermediate layers of this case is 6. The x₁ to x₁₀ of the input layer corresponding to the 1st-layer are as shown in Table 3, i=1 to 10, hu 1 to hu 6 correspond to respective units of the intermediate layer, and j=1 to 6.

TABLE 3

| | | | |
|---|---|---|---|
| x1 | Age years | | |
| x2 | Sex | male: 1 | female: 2 |
| x3 | Cerebrovascular accidents | absent: 0 | present: 1 |
| x4 | Malignant diseases | absent: 0 | present: 1 |
| x5 | Deglutition pneumonia | absent: 0 | present: 1 |
| | before gastrostomy | | |
| x6 | Dementia | absent: 0 | present: 1 |
| x7 | Degenerative diseases | absent: 0 | present: 1 |
| x8 | Serum total protein | g/dl | |
| x9 | Serum albumin | g/dl | |
| x10 | Hemoglobin | g/dl | |

Next, the output y1 is predicted by propagating the information using the matrix W_{p,q} shown in the following Expression (3), based on the results obtained from formulae (1) and (2). The calculation in the 2nd-layer shown in Table 4 is performed based on the following calculating expressions. Each unit in the intermediate layer and output layer synthesizes the input factors from the intermediate layer by the following Expression (3) and outputs the sigmoid function of Expression (4) (corresponding to the prognosis prediction expression which is described later) into the output layer as an operation function (step 23). However, the following table is an example of the learning step.

$$\begin{matrix}{y}_{j} = \sum {w}_{p , q} {x}_{p} & ( 3 ) \\ f ( {y}_{j} ) = \frac{1}{1 + exp ( - α {y}_{j} )} & ( 4 )\end{matrix}$$

TABLE 4

| 2nd-layer | hu 1 | hu 2 | hu 3 | hu 4 | hu 5 | hu 6 |
|---|---|---|---|---|---|---|
| y1 | 76.13407 | −22.8014 | −10.6726 | 12.15486 | −13.7676 | −15.4048 |

In the above description, relationship between prediction input factors and prediction output factors of two or more patients stored in the prediction factor data base 12 was patternized using age, sex, the presence or absence of cerebrovascular accidents, the presence or absence of malignant diseases, the presence or absence of deglutition pneumonia before gastrostomy, the presence or absence of dementia, the presence or absence of degenerative diseases, amount of serum total protein, amount of serum albumin and amount of hemoglobin as the prediction input factors and using the number of the number of survival days after PEG as the prediction output factors. Hereinafter, the sigmoid function output from the intermediate layer to the output layer via the above-mentioned process is called prognosis prediction formula. When the prognosis prediction expression is calculated by performing the program extracted in the analytical program storage section 13, the processing section 16 stores said prognosis prediction expression in the prognosis prediction expression storage section 14 (step 24). A series of these processings of reading out the prediction factors stored in the prediction factor data base 12 (step 21), calculating the prognosis prediction expression (steps 22 and 23 and storing the calculated prognosis prediction expression in the prognosis prediction expression storage section 14 (step 24) is referred sometimes to as patternizing phase.

In this connection, when other factor, for example, the presence or absence of the onset of deglutition pneumonia after PEG, is used as the prediction output factor, it can be patternized using the numerical values shown in Tables 5 and 6. However, in the case of the presence or absence of the onset of deglutition pneumonia after PEG, the number of units in the intermediate layer is 5.

TABLE 5
outcome y2
n(hidden) 5

| 1st-layer | x1 | x2 | x3 | x4 | x5 |
|---|---|---|---|---|---|
| hu 1 | −2.3085 | 5.262328 | −3.42721 | −2.50708 | 8.378059 |
| hu 2 | 3.421257 | 1.734852 | 3.842312 | −0.9846 | −7.38805 |
| hu 3 | 3.408629 | −1.23626 | −2.64124 | 2.143445 | 3.505778 |
| hu 4 | 2.260281 | 3.89501 | 2.965592 | −4.37115 | 4.976577 |
| hu 5 | 6.213096 | −19.1835 | 4.622625 | −3.21563 | 1.545865 |
| | x6 | x7 | x8 | x9 | x10 |
| hu 1 | −4.16963 | −0.04007 | −1.79303 | −0.51161 | 4.204196 |
| hu 2 | −0.25455 | −3.62649 | 5.009584 | −2.13615 | 1.975483 |
| hu 3 | −6.71591 | 2.009129 | 5.705797 | 3.250639 | −0.77613 |
| hu 4 | −0.26223 | 2.347613 | −10.5205 | 0.279854 | −3.00052 |
| hu 5 | 11.94504 | 1.052677 | 6.119155 | 4.077493 | 5.303127 |

TABLE 6

| 2nd-layer | hu 1 | hu 2 | hu 3 | hu 4 | hu 5 |
|---|---|---|---|---|---|
| y2 | 10.67555 | −12.4321 | −11.6476 | 21.27991 | −1.41908 |

The structure of ANN can be optionally set, but a three layer type ANN in which each of the input layer, intermediate layer and output layer consists of one layer is general. By increasing the number of units in the intermediate layer, approximation of further complex functions becomes possible. However, when it is too easily increased, over-learning occurs so that ANN starts to cause unnatural prediction. In order to avoid this, it is necessary to set the necessary minimum number of units. Though a universal method for determining the number of units in intermediate layer has not been developed yet, there is broadly used a method in which the prediction accuracy is checked by separately preparing test data for evaluating predictability of ANN, and thereby selecting an ANN structure which produces minimum prediction error. A leave-one-out method is used in the prediction device of the embodiment of the invention. This is a method in which ANN is caused to perform learning by leaving a pair of data for evaluation use from leaning data, the same operation is carried out thereafter by changing the data for evaluation use one by one, and an ANN structure wherein the sun total of prediction errors of the evaluation data becomes minimum is selected.

Next, processings after the patternizing phase are described. A relationship between input factors and output factors is patternized by the prognosis prediction expression calculated in the patternizing phase. Thus, when a doctor judges whether or not to perform PEG for a patient, diagnosis input factors, as the same items of the prediction input factors which were used as references in calculating a prognosis prediction expression in the patternizing phase, are input by operating the Input section 11. Then, the processing section 16 calculates diagnosis output factors, by substituting the diagnosis input factors input by the Input section 11 for the prognosis prediction expression stored in the prognosis prediction expression storage section 14, and outputs the calculated diagnosis output factors by the display section 15. A series of processings of inputting diagnosis input factors of a patient for judging whether or not to perform PEG, calculating diagnosis output factors based on the prognosis prediction expression and outputting the calculated diagnosis output factors is referred sometimes to as diagnosis phase.

A doctor judges, in the diagnosis phase, whether or not to perform PEG for the above-mentioned patient, based on the diagnosis output factors output onto the display section 15 such as the number of survival days and a possibility of causing onset of deglutition pneumonia in the case of performing PEG.

Thus, according to the prediction device of the embodiment of the invention, the number of survival days of a patient after PEG and a possibility of a complication with the disease which caused a disorder in deglutition function can be predicted with a high accuracy, by predicting prognosis of a patient who underwent PEG based on two or more prediction factors. As a result of this, a doctor can be informed with a prediction result which is sufficient enough for judging whether or not to perform PEG for the patient.

In this connection, according to the embodiment of the invention, description has been given on a case in which the number of survival days after PEG or the presence or absence of the onset of deglutition pneumonia after PEG is output as a diagnosis output factor. This is because these two diagnosis output factors are factors which are regarded important by doctors in judging whether or not to perform PEG. In order to calculate these two diagnosis output factors with high accuracy, it is desirable to use, as prediction input factors, age, sex, the presence or absence of a cerebrovascular accident, the presence or absence of a malignant disease, the presence or absence of deglutition pneumonia before the gastrostomy, the presence or absence of dementia, the presence or absence of a degenerative disease, amount of serum total protein, amount of serum albumin and amount of hemoglobin.

While the invention has been described in detail and with reference to specific embodiments thereof, it will be apparent to one skilled in the art that various changes and modifications can be made therein without departing from the spirit and scope of the invention.

This application is based on a Japanese patent application filed on May 7, 2008 (Japanese Patent Application No. 2008-121326), the entire contents thereof being thereby incorporated by reference.

### [FIG. 1]

11: Input section (keyboard),
12: Prediction factor data base (HDD),
13: Analytical program storage section (first memory),
14: Prognosis prediction formula storage section (second memory),
15: Display section (display),
16: Processing section (CPU)

### [FIG. 2]

#1: Start of patternizing phase,
S21: Input prediction factors into input layer,
S22: Synthesize input prediction factors and output sigmoid function into intermediate layer,
S23: Synthesize input prediction factors and output prognosis prediction expression into output layer,
S24: Store prognosis prediction expression,
#2: End patternizing phase,
#3: Input layer,
#4 Intermediate layer,
#5 Output layer

1. A device that predicts prognosis of a patient who undergoes PEG (percutaneous endoscopic gastrostomy), the device comprising:
a prognosis prediction expression storage section configured to store a prognosis prediction expression, said prognosis prediction expression being calculated by applying prediction input factors and prediction output factors about a first patient who already underwent the PEG, to ANN (artificial neural network); and
a processing section configured to:
receive diagnosis input factors about a second patient, for judging whether or not to perform the PEG on the second patient;
calculate diagnosis output factors in response to the diagnosis input factors based on the prognosis prediction expression; and
outputs the calculated diagnosis output factors.

2. The device according to claim 1, further comprising:
a prediction factor data base configured to store the prediction input factors and the prediction output factors; and
an analytical program storage section configured to store a program for calculating the prognosis prediction expression, and
wherein
the processing section calculates the prognosis prediction expression based on the program, and then stores the calculated prognosis prediction expression in the prognosis prediction expression storage section.

3. The device according to claim 1, wherein the prediction output factors and the diagnosis output factors include at least one of the number of survival days after the PEG and, presence or absence of onset of deglutition pneumonia after the PEG.

4. The device according to claim 3, wherein the prediction input factors and the diagnosis input factors includes at least age, sex, the presence or absence of a cerebrovascular accident, the presence or absence of a malignant disease, the presence or absence of deglutition pneumonia before gastrostomy, the presence or absence of dementia, the presence or absence of a degenerative disease, an amount of serum total protein, an amount of serum albumin and an amount of hemoglobin.

5. A method for predicting prognosis of a patient who undergoes PEG (percutaneous endoscopic gastrostomy), the method comprising:
inputting diagnosis input factors about a first patient, for judging whether or not to perform the PEG on the first patient;
calculating diagnosis output factors in response to the diagnosis input factors, based on a prognosis prediction expression; and
outputting the diagnosis output factors.

6. The method according to claim 5, further comprising:
calculating the prognosis prediction expression by applying prediction input factors and prediction output factors about a second patient who already underwent the PEG to ANN.

7. A computer readable medium storing a program causing a computer to execute the method according to claim 5.
